(19) United States
(12) Patent Application Publication
(10) Pub. No.: US 2024/0316222 A1
(43) Pub. Date: Sep. 26, 2024

(54) POLYMER MICROSPHERE AND PREPARATION METHOD THEREOF

(71) Applicants: BEIJING BOE TECHNOLOGY DEVELOPMENT CO., LTD., Beijing (CN); BOE Technology Group Co., Ltd., Beijing (CN)

(72) Inventors: Zhihong Wu, Beijing (CN); Ding Ding, Beijing (CN)

(73) Assignees: BEIJING BOE TECHNOLOGY DEVELOPMENT CO., LTD., Beijing (CN); BOE Technology Group Co., Ltd., Beijing (CN)

(21) Appl. No.: 18/638,130

(22) Filed: Apr. 17, 2024

Related U.S. Application Data
(63) Continuation of application No. PCT/CN2023/082866, filed on Mar. 21, 2023.

(51) Int. Cl. A61K 49/18; A61K 9/50

(52) U.S. Cl. CPC A61K 49/1854; A61K 9/5089

(57) ABSTRACT

The present application provides a polymer microsphere and a preparing method thereof. The polymer microsphere includes a core and a shell coating the core; the core includes at least two magnetic particles and a dispersion located between two neighboring magnetic particles; the shell includes a main shell body and a modifying layer, and the modifying layer is located on one side of the main shell body that is away from the magnetic particles; and a material of the dispersion, a material of the main shell body and a material of the modifying layer are the same. The magnetic particles in the polymer microsphere have a good dispersibility, and, when heated, the magnetic attraction force and the magnetic responsiveness of the polymer microsphere can nearly maintain unchanged.

### TECHNICAL FIELD

The present application relates to the field of biomedicine, and particularly relates to a polymer microsphere and a preparing method thereof.

### BACKGROUND

Magnetic nanometer or micrometer composite materials, for example, magnetic microspheres, have been paid extensive attention due to their potential applications in the field of biomedicine. The magnetic microspheres in the related art, because of the restrictions by the sizes and the thermal stability, have the problem of a poor effect of magnetic response or an unstable magnetic adsorption force in practical applications. Currently, it is urgently needed to provide a novel magnetic microsphere to solve the above problems.

### SUMMARY

The embodiments of the present application employ the following technical solutions:

In the first aspect, an embodiment of the present application provides a polymer microsphere, wherein the polymer microsphere comprises a core and a shell coating the core;
the core comprises at least two magnetic particles and a dispersion located between two neighboring instances of the magnetic particles;
the shell comprises a main shell body and a modifying layer, and the modifying layer is located on one side of the main shell body that is away from the magnetic particles; and
a material of the dispersion, a material of the main shell body and a material of the modifying layer are the same.

In at least one embodiment of the present application, the dispersion covers at least part of a surface of the magnetic particles.

In at least one embodiment of the present application, each of the material of the dispersion, the material of the main shell body and the material of the modifying layer comprises a polymer formed by at least one first monomer and at least one second monomer; and a range of a ratio of a molar weight of the first monomer to a molar weight of the second monomer is 0.1-10.

In at least one embodiment of the present application, each of the second monomer and the modifying layer comprises at least one of amino, carboxyl, hydroxyl and silicon hydroxyl.

In at least one embodiment of the present application, the molar weight of the second monomer is less than the molar weight of the first monomer.

In at least one embodiment of the present application, the first monomer comprises at least one of styrene, butadiene, pentadiene, isoprene, methyl propenyl ketone, acrylonitrile, methyl methacrylate and tetraethyl orthosilicate; and
the second monomer comprises at least one of methacrylic acid, ethyleneimine, hydroxyethylene propylene, ethyl orthosilicate, vinyl siloxane, N-(4-carboxylphenyl)maleimide, aminopropyltriethoxysilane and glyoxal.

In at least one embodiment of the present application, a range of a ratio of a mass of the magnetic particles to a sum of a mass of the first monomer and a mass of the second monomer is 0.5-3.

In at least one embodiment of the present application, the first monomer comprises styrene, the second monomer comprises methacrylic acid, and the range of the ratio of the molar weight of the first monomer to the molar weight of the second monomer is 0.1-10.

In at least one embodiment of the present application, the first monomer comprises tetraethyl orthosilicate, the second monomer comprises aminopropyltriethoxysilane and glyoxal, a range of a ratio of the molar weight of the first monomer to a molar weight of a total monomer amount is 0.1-0.8, a range of a ratio of a molar weight of the aminopropyltriethoxysilane in the second monomer to the molar weight of the total monomer amount is 0.1-0.4, and a range of a ratio of a molar weight of the glyoxal in the second monomer to the molar weight of the total monomer amount is 0.1-0.5.

In at least one embodiment of the present application, the first monomer comprises styrene, the second monomer comprises ethyleneimine, a range of a ratio of the molar weight of the first monomer to a molar weight of a total monomer amount is 0.1-0.6, and a range of a ratio of the molar weight of the second monomer to the molar weight of the total monomer amount is 0.4-0.9.

In at least one embodiment of the present application, a range of a distance from each of the magnetic particles to the modifying layer in a direction from a geometric center of the polymer microsphere pointing to the modifying layer is 10 nm-500 nm.

In at least one embodiment of the present application, the magnetic particles comprise one or more of an iron oxide, a cobalt oxide and a nickel oxide, and a range of a size of each of the magnetic particles is 8 nm-15 nm.

In at least one embodiment of the present application, a range of a size of the polymer microsphere is 100 nm-5 μm.

In the second aspect, an embodiment of the present application provides a preparing method of a polymer microsphere, wherein the preparing method is applied to prepare the polymer microsphere according to any one of the embodiments in the first aspect, and the method comprises:
forming the magnetic particles; and
mixing the magnetic particles, at least one first monomer and at least one second monomer together, to form the polymer microsphere in one step by in-situ polymerization, wherein the polymer microsphere comprises the core and the shell coating the core; the core comprises at least two instances of the magnetic particles and a dispersion located between two neighboring instances of the magnetic particles; the shell comprises a main shell body and a modifying layer, and the modifying layer is located on one side of the main shell body that is away from the magnetic particles; and all of the dispersion, the main shell body and the modifying layer have the same materials and comprise a polymer formed by at least one instance of the first monomer and at least one instance of the second monomer.

In at least one embodiment of the present application, the step of mixing the magnetic particles, the at least one first monomer and the at least one second monomer together, to form the polymer microsphere in one step by in-situ polymerization comprises:
mixing the magnetic particles, one instance of the first monomer and one instance of the second monomer together, to form the polymer microsphere in one step by in-situ polymerization; or
mixing the magnetic particles, one instance of the first monomer and two instances of the second monomer together, to form the polymer microsphere in one step by in-situ polymerization.

In at least one embodiment of the present application, the step of mixing the magnetic particles, the one instance of the first monomer and the one instance of the second monomer together, to form the polymer microsphere in one step by in-situ polymerization comprises:
dispersing the magnetic particles in anhydrous ethanol, to obtain a suspension of the magnetic particles;
adding polyvinylpyrrolidone, an initiator and the second monomer into the suspension of the magnetic particles, and introducing a protective gas; and after stirring uniformly, adding the first monomer, and stirring continuously at a first preset temperature to react for 2-6 hours, wherein the first monomer comprises styrene, and the second monomer comprises methacrylic acid; and
after reaction liquid has cooled to room temperature, washing by using anhydrous ethanol and deionized water, and drying to obtain the polymer microsphere.

In at least one embodiment of the present application, the step of mixing the magnetic particles, the one instance of the first monomer and the one instance of the second monomer together, to form the polymer microsphere in one step by in-situ polymerization comprises:
dispersing the magnetic particles in anhydrous ethanol, to obtain a suspension of the magnetic particles;
adding polyvinylpyrrolidone, an initiator, the first monomer and the second monomer into the suspension of the magnetic particles; and stirring continuously under vacuum at a second preset temperature to react for 2-4 hours, wherein the first monomer comprises styrene, and the second monomer comprises ethyleneimine; and
after reaction liquid has cooled to room temperature, washing by using anhydrous ethanol and deionized water, and drying to obtain the polymer microsphere.

In at least one embodiment of the present application, the step of mixing the magnetic particles, the one instance of the first monomer and the two instances of the second monomer together, to form the polymer microsphere in one step by in-situ polymerization comprises:
dispersing the first monomer in anhydrous ethanol;
dispersing the magnetic particles in a water solution comprising polyvinylpyrrolidone, to obtain a suspension of the magnetic particles;
adding the suspension of the magnetic particles into the anhydrous ethanol in which the first monomer is dispersed, subsequently adding the two second monomers and ammonia, stirring uniformly, and subsequently reacting at a third preset temperature for 1-3 hours, wherein the first monomer comprises tetraethyl orthosilicate, and the second monomer comprises aminopropyltriethoxysilane and glyoxal; and
after reaction liquid has cooled to room temperature, washing by using anhydrous ethanol and deionized water, and drying to obtain the polymer microsphere.

In at least one embodiment of the present application, the step of forming the magnetic particles comprises:
mixing ferric trichloride hydrate, ferrous chloride hydrate and isopropanol together, and stirring uniformly;
heating to 50° C.±5° C., dripping concentrated ammonia slowly, and stirring to react for 10-30 minutes;
after reaction ends, standing reaction liquid at 0-10° C. for 4-6 hours;
washing by using methanol, adding a methanol solution of oleic acid, and stirring for 2-5 hours, to obtain the magnetic particles; and
re-washing by using methanol, and dispersing the obtained magnetic particles into hexane.

In at least one embodiment of the present application, the step of forming the magnetic particles comprises:
mixing ferric trichloride, ferrous chloride, hydrochloric acid and deionized water together, and stirring uniformly, to obtain an iron-ion mixture liquid;
introducing a protective gas, dripping the iron-ion mixture liquid into a sodium-hydroxide solution at 80° C.±5° C. under continuous stirring, and after the iron-ion mixture liquid is dripped completely, reacting for 20-40 minutes; and
after reaction ends, cooling to room temperature, washing by using anhydrous ethanol and deionized water, and drying to obtain the magnetic particles.

The above description is merely a summary of the technical solutions of the present application. In order to more clearly know the elements of the present application to enable the implementation according to the contents of the description, and in order to make the above and other purposes, features and advantages of the present application more apparent and understandable, the particular embodiments of the present application are provided below.

### BRIEF DESCRIPTION OF THE DRAWINGS

In order to more clearly illustrate the technical solutions of the embodiments of the present application or the related art, the figures that are required to describe the embodiments or the related art will be briefly described below. Apparently, the figures that are described below are merely embodiments of the present application, and a person skilled in the art can obtain other figures according to these figures without paying creative work.

FIG. 1 and FIG. 2 are the structures of two types of polymer microspheres in the related art;

FIG. 3 is a structure of a polymer microsphere according to an embodiment of the present application;

FIG. 4 is a flow chart of a preparing method of a polymer microsphere according to an embodiment of the present application;

FIG. 5 to FIG. 7 are diagrams for describing the principle of the preparation of a first type of the polymer microsphere according to embodiments of the present application;

FIG. 8 and FIG. 9 are diagrams for describing the principle of the preparation of a second type of the polymer microsphere according to embodiments of the present application;

FIG. 10 and FIG. 11 are diagrams for describing the principle of the preparation of a third type of the polymer microsphere according to embodiments of the present application; and

FIG. 12 to FIG. 15 are diagrams for describing the comparison of the effects of the magnetic attraction force of the polymer microspheres according to the embodiments of the present application after heating.

### DETAILED DESCRIPTION

The technical solutions of the embodiments of the present application will be clearly and completely described below with reference to the drawings of the embodiments of the present application. Apparently, the described embodiments are merely certain embodiments of the present application, rather than all of the embodiments. All of the other embodiments that a person skilled in the art obtains on the basis of the embodiments of the present application without paying creative work fall within the protection scope of the present application.

Unless the context otherwise requires, in the entire specification and claims, the term “including/comprising” is interpreted as open and inclusive, meaning “including, but not limited to.”. In the description of the specification, the terms “one embodiment,” “some embodiments,” “exemplary embodiments,” “examples,” “specific examples,” or “some examples,” etc. are intended to indicate that specific features, structures, materials, or features related to the embodiment or example are included in at least one embodiment or example of the present application. The schematic representation of the above terms may not necessarily refer to the same embodiment or example. Furthermore, the specific features, structures, materials, or features described may be included in any one or more embodiments or examples in any appropriate manner.

In the embodiments of the present application, the use of words such as “first” and “second” to partially describe identical or similar items with similar functions and effects is only for the purpose of clearly describing the technical solution of the embodiments of the present application, and cannot be understood as indicating or implying relative importance or implying the number of indicated technical features.

Magnetic nanometer or micrometer composite materials, for example, magnetic beads, have been paid extensive attention due to their potential applications in the fields of biotechnology and biomedicine. In the field of biology, magnetic beads are usually classified according to the uses, and the magnetic beads of different uses differ in the sizes, the surface modifying layers, the storage modes and the synthesizing methods. Magnetic beads are currently applied in the field of biology in nucleic acid extraction, chemiluminiscence, cell sorting, protein purification, biological catalysis, fluorescence labeling, magnetic resonance imaging, magnetothermal therapy and so on. The magnetic beads in the related art, because of the restrictions by the sizes and the thermal stability, have the problem of a poor effect of magnetic response or an unstable magnetic adsorption force in practical applications.

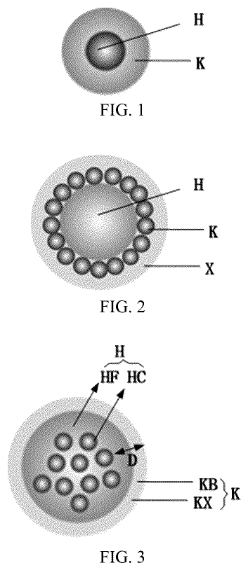

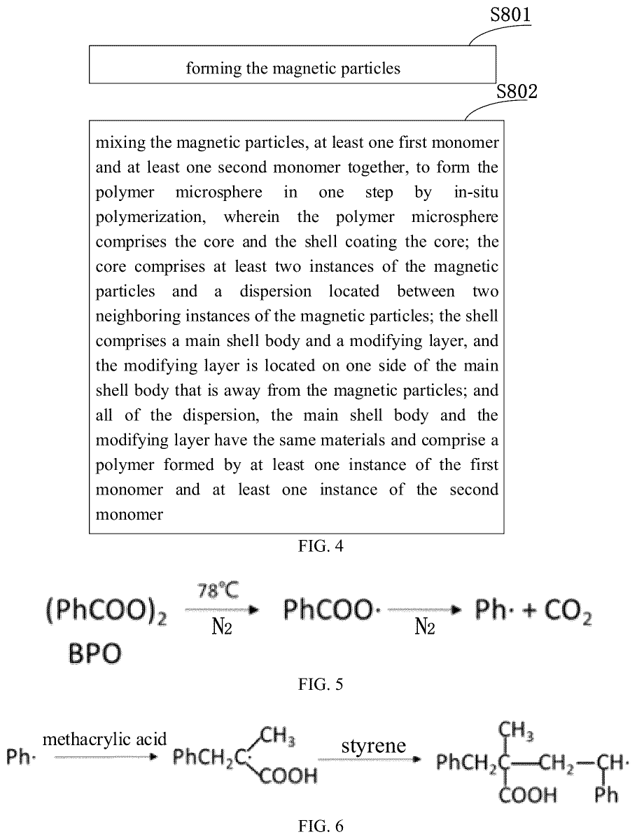

For example, a case in the related art is that magnetic particles are embedded in a polymer microsphere, wherein the magnetic particles serve as the core structure of the polymer microsphere, and a high polymer serves as the shell structure. However, in the magnetic polymer microsphere formed in such a mode, as shown in FIG. 1, when the magnetic particles are, as the core structure H, embedded in the polymer microsphere (the shell structure K), because of the restriction by the size of the polymer microsphere, the magnetic particles have a low overall content, and a poor effect of magnetic response. It should be noted that the restriction by the size of the polymer microsphere refers to that, in the polymerization processes in the related art, the range of the sizes of the polymer microspheres is usually from hundreds of nanometers to several micrometers, and that of the magnetic particles is usually approximately 10 nm, which causes that the overall content of the magnetic particles in the polymer microsphere is very low.

As another example, another case in the related art is that, as shown in FIG. 2, a polymer microsphere serves as the core structure H, at least one layer of magnetic particles coats the outer side of the polymer microsphere to serve as the shell structure K, and subsequently a modifying layer X is formed on the magnetic particles serving as the shell structure K, to protect the magnetic particles. However, in such a structure, after the magnetic particles have been circularly heated, the interfaces of the magnetic particles interfuse, which causes that the coercive force changes, to cause the magnetic response and the magnetic attraction force to be unstable.

The coercive force refers to that, after saturation magnetization of a magnetic material, when the external magnetic field has decreased to zero, its magnetic induction intensity does not decrease to zero, and merely when a magnetic field of a certain magnitude is added in the direction opposite to the original magnetizing field, the magnetic induction intensity can be reduced to zero, wherein that magnetic field is referred to as a coercive field, and is also referred to as a coercive force.

In view of that, an embodiment of the present application provides a polymer microsphere, wherein the polymer microsphere comprises a core and a shell coating the core; the core comprises at least two magnetic particles and a dispersion located between two neighboring magnetic particles; the shell comprises a main shell body and a modifying layer, and the modifying layer is located on the side of the main shell body that is away from the magnetic particles; and the material of the dispersion, the material of the main shell body and the material of the modifying layer are the same. In the embodiments of the present application, the polymer microsphere is synthesized by means of in-situ polymerization. The core structure in the polymer microsphere comprises the magnetic particles and the dispersion, and when the quantity of the magnetic particles in the core structure is two or more, the dispersion facilitates to separate the interfaces between the magnetic particles, which prevents that, in the case of thermal cycle, the interfaces of the magnetic particles in the core structure interfuse. The shell structure of the polymer microsphere comprises the main shell body and the modifying layer, wherein the main shell body serves to protect the core structure, and the modifying layer causes the surface of the shell to have particular functional groups. By configuring that the material of the dispersion, the material of the main shell body and the material of the modifying layer are the same, that can, to a large extent, simplify the configuring of the shell structure and simplify the preparing process of the polymer microsphere, thereby reducing the cost while ensuring its quality.

The embodiments of the present application will be described below with reference to the drawings.

An embodiment of the present application provides a polymer microsphere. As shown in FIG. 3, the polymer microsphere comprises a core H and a shell K coating the core H.

The core H comprises at least two magnetic particles HC and a dispersion HF located between two neighboring magnetic particles HC. The shell K comprises a main shell body KB and a modifying layer KX, and the modifying layer KX is located on the side of the main shell body KB that is away from the magnetic particles HC. The material of the dispersion HF, the material of the main shell body KB and the material of the modifying layer KX are the same.

The material of the magnetic particles HC is not limited herein. As an example, the material of the magnetic particles HC may comprise a magnetic material.

For example, the magnetic material may be an alloy-type material, and the main types of the alloy include AlNi(Co), FcCr(Co), FeCrMo, FeAIC, FeCo(V)(W), Re-Co (Re represents rare-earth elements), Re-Fe, AlNi(Co), FcCrCo, FeCrCo, PtCo, MnAIC, CuNiFc, AlMnAg and so on.

As another example, the magnetic material may comprise a ferrite-type material, whose main component is MO·6Fe₂O₃, wherein M represents Ba, Sr, Pb or a composite component such as SrCa and LaCa.

As another example, the magnetic material may be an intermetallic compound, which is represented mainly by MnBi.

The magnetic particles HC according to the embodiments of the present application will be described by taking the case as an example in which their main component is ferric oxide (Fe₃O₄) and/or ferrous oxide (Fe₂O₃).

In an exemplary embodiment, the dispersion HF is used to disperse and isolate the magnetic particles HC, to prevent that, in the situation of thermal cycle, two neighboring magnetic particles HC have interface interfusion therebetween.

In at least one embodiment of the present application, the dispersion HF is configured to be capable of coating at least part of the surfaces of the magnetic particles HC. That the dispersion HF is configured to be capable of coating at least part of the surfaces of the magnetic particles HC includes but is not limited to the following cases:

In the first case, all of the magnetic particles HC are coated separately by the dispersion HF, in which case the dispersion HF is configured to be capable of coating all of the surfaces of the magnetic particles HC.

In the second case, regarding all of the magnetic particles HC, two or more magnetic particles HC that are coated together by the dispersion HF exist, in which case the dispersion HF is configured to be capable of coating part of the surfaces of the magnetic particles HC.

In the third case, all of the magnetic particles HC include a first part and a second part. The first part of the magnetic particles HC are coated separately by the dispersion HF, and the dispersion HF is configured to be capable of coating all of the surfaces of the first part of the magnetic particles HC. In the second part of the magnetic particles HC, two or more magnetic particles HC are coated together by the dispersion HF, and the dispersion HF is configured to be capable of coating part of the surfaces of the magnetic particles HC.

As an example, the main shell body KB and the modifying layer KX are of an integral structure, wherein the integral structure means being prepared by using the same materials in the same one preparing process. It can be understood that the main shell body KB and the modifying layer KX may be wholly deemed as one component.

As an example, the dispersion HF, the main shell body KB and the modifying layer KX are of an integral structure.

In an exemplary embodiment, the modifying layer HX includes functionalizing functional groups, for example, at least one of amino, carboxyl, hydroxyl and silicon hydroxyl, so that the surface of the polymer microsphere has at least one of those functionalizing functional groups.

In the embodiments of the present application, because the material of the dispersion HF, the material of the main shell body KB and the material of the modifying layer KX are the same, the dispersion HF and the main shell body KB also include the functionalizing functional groups, for example, at least one of amino, carboxyl, hydroxyl and silicon hydroxyl.

In an exemplary embodiment, it may be configured that the material of the dispersion HF, the main shell body KB and the modifying layer KX is a copolymer, which is also referred to as a copolymer body. A copolymer is a polymer obtained by the addition polymerization reaction of two or more different monomers. According to the different arrangement modes of the monomers in the molecular chains of copolymers, copolymers may be classified into random copolymer, alternating copolymer, block copolymer and graft copolymer. The type of the copolymer is not limited in the present application.

In an exemplary embodiment, it may be configured that the material of the dispersion HF, the main shell body KB and the modifying layer KX is a polycondensate. A polymer formed by the condensation polymerization reaction of two monomers including different groups is referred to as a polycondensate.

A copolymer refers to a polymer obtained by an addition polymerization reaction, and a polycondensate refers to a polymer formed by a condensation polymerization reaction, wherein the reaction mechanisms of them are different. The particular differences of addition polymerization and condensation polymerization may refer to the description in the related art, and are not discussed further herein.

In the embodiments of the present application, the polymer microsphere is synthesized by means of in-situ polymerization. The core structure H in the polymer microsphere comprises the magnetic particles HC and the dispersion HF, and when the quantity of the magnetic particles HC in the core structure H is two or more, the dispersion HF facilitates to separate the interfaces between the magnetic particles HC, which prevents that, in the case of thermal cycle, the interfaces of the magnetic particles HC in the core structure H interfuse. The shell structure K of the polymer microsphere comprises the main shell body KB and the modifying layer KX, wherein the main shell body KB serves to protect the core structure H, and the modifying layer KX causes the surface of the shell K to have particular functional groups. By configuring that the material of the dispersion HF, the material of the main shell body KB and the material of the modifying layer KX are the same, that can, to a large extent, simplify the configuring of the shell structure K and simplify the preparing process of the polymer microsphere, thereby reducing the cost while ensuring its quality.

In at least one embodiment of the present application, the polymer microsphere may be used for the extraction of nucleic acids, wherein the nucleic acids include ribonucleic acids (referred to for short as RNA) and deoxyribonucleic acids (referred to for short as DNA).

In at least one embodiment of the present application, the polymer microsphere may be used for the extraction and the purification of antibody proteins.

In at least one embodiment of the present application, each of the material of the dispersion HF, the material of the main shell body KB and the material of the modifying layer KX comprises a polymer formed by at least one first monomer and at least one second monomer. The range of the ratio of the molar weight of the first monomer to the molar weight of the second monomer is 0.1-10.

As an example, the first monomer may serve as the main-body monomer, to provide the matrix of the polymer microsphere. The second monomer may serve as the functionalizing monomer, to provide the functionalizing functional groups, wherein the functionalizing functional groups include but are not limited to at least one of amino, carboxyl, hydroxyl and silicon hydroxyl.

In at least one embodiment of the present application, each of the second monomer and the modifying layer comprises at least one of amino, carboxyl, hydroxyl and silicon hydroxyl.

The quantity of the type of the first monomer and the quantity of the type of the second monomer are not limited herein. As an example, in order to facilitate the polymerization reaction and simplify the polymerization preparing process, it may be configured that each of the material of the dispersion HF, the material of the main shell body KB and the material of the modifying layer KX comprises a polymer formed by one first monomer and at least one second monomer.

The range of the ratio of the molar weight of the first monomer to the molar weight of the second monomer is 0.1-10. The molar weight is actually also referred to as molar mass. The mass per mole of a substance is referred to as its “molar mass”.

As an example, the ratio of the molar weight of the first monomer to the molar weight of the second monomer may be 0.2, 0.3, 0.4, 0.5, 0.6, 0.7, 0.8, 0.9, 1.0, 1.1, 1.5, 1.8, 3, 3.5, 4, 4.5, 5, 5.5, 6, 6.5, 7, 7.5, 8, 8.5, 9 or 9.5.

If the ratio of the molar weight of the first monomer to the molar weight of the second monomer is greater than 1, the proportion of the molar mass of the first monomer is greater than the proportion of the molar mass of the second monomer.

In at least one embodiment of the present application, the molar weight of the second monomer is less than the molar weight of the first monomer.

In the embodiments of the present application, by configuring that the molar weight of the second monomer is less than the molar weight of the first monomer, the contents of the functionalizing functional groups provided by the functionalizing monomer can be effectively controlled and adjusted. In an aspect, that can effectively reduce the problem of agglomeration of the polymer microsphere caused by a too high concentration of the functionalizing functional groups, and in another aspect, that can prevent the problem to a large extent that two neighboring functionalizing functional groups on the same one polymer microsphere have a chemical reaction therebetween to result in failure of the functionalizing functional groups on the surface of the polymer microsphere, thereby improving the storage stability of the polymer microsphere, and prolonging the storage period of the polymer microsphere.

In at least one embodiment of the present application, the first monomer comprises at least one of styrene, butadiene, pentadiene, isoprene, methyl propenyl ketone, acrylonitrile, methyl methacrylate and tetraethyl orthosilicate; and

the second monomer comprises at least one of methacrylic acid, ethyleneimine, hydroxyethylene propylene, ethyl orthosilicate, vinyl siloxane, N-(4-carboxylphenyl)maleimide, aminopropyltricthoxysilane and glyoxal.

In the description, comprising at least one of A, B and C refers to comprising one of A, B and C, or a combination of more than one of A, B and C. “Plurality of” refers to “two or more”. The meanings of the similar recitations at other positions are similar to the above-described situation, and are not discussed further below.

In at least one embodiment of the present application, the range of the ratio of the mass of the magnetic particles HC to the sum of the mass of the first monomer and the mass of the second monomer is 0.5-3.

The ratio of the mass of the magnetic particles HC to the sum of the mass of the first monomer and the mass of the second monomer may be understood as the ratio of the mass of the magnetic particles HC to the sum of the mass of the shell K and the mass of the dispersion HF.

In an exemplary embodiment, in order to ensure that the polymer microsphere has a stable and accurate magnetic responsiveness, there are requirements on the mass of the magnetic particles HC.

As an example, the ratio of the mass of the magnetic particles HC to the sum of the mass of the first monomer and the mass of the second monomer may be 0.6, 0.7, 0.8, 0.9, 1.0, 1.2, 1.4, 1.5, 1.8, 2.0, 2.3, 2.5, 2.8 or 2.9. If the numerical value of that ratio is high, the proportion of the magnetic particles HC in the polymer microsphere is higher, and the polymer microsphere has a better effect of magnetic response.

It should be noted that, if the ratio of the mass of the magnetic particles HC to the sum of the mass of the first monomer and the mass of the second monomer is higher, the shell K of the polymer microsphere has a larger thickness. If the ratio of the mass of the magnetic particles HC to the sum of the mass of the first monomer and the mass of the second monomer is greater than 3, the magnetic responsiveness of the polymer microsphere might decrease, which reduces the accuracy of the detection of the polymer microsphere in the field of biomedicine, thereby reducing its usage efficiency. If the ratio of the mass of the magnetic particles HC to the sum of the mass of the first monomer and the mass of the second monomer is less than 0.5, the thickness of the shell K of the polymer microsphere is too small, and by the effect of an external force, the shell K is insufficient to protect the magnetic particles HC in the core structure H, and cracking of the shell structure K very easily happens. By the effect of thermal cycle, the shell K is insufficient to reduce the heat received by the magnetic particles HC, and the problem of interface interfusion of the magnetic particles HC very easily happens, which reduces the magnetic responsiveness, and changes the coercive force.

In at least one embodiment of the present application, the first monomer comprises styrene, the second monomer comprises methacrylic acid, and the range of the ratio of the molar weight of the first monomer to the molar weight of the second monomer is 0.1-10.

As an example, the ratio of the molar weight of the first monomer to the molar weight of the second monomer may be 0.2, 0.3, 0.4, 0.5, 0.6, 0.7, 0.8, 0.9, 1.0, 1.1, 1.5, 1.8, 3, 3.5, 4, 4.5, 5, 5.5, 6, 6.5, 7, 7.5, 8, 8.5, 9 or 9.5.

In the case that the first monomer comprises styrene, and the second monomer comprises methacrylic acid, the copolymer formed by the first monomer and the second monomer is a copolymer of styrene and methacrylic acid. Because the second monomer is methacrylic acid, the surface (the modifying layer KX) of the polymer microsphere finally has carboxyl (—COOH).

When the surface (the modifying layer KX) of the polymer microsphere has carboxyl (—COOH), the polymer microsphere may be used for the extraction and the purification of DNA.

In at least one embodiment of the present application, the first monomer comprises tetraethyl orthosilicate, the second monomer comprises aminopropyltriethoxysilane and glyoxal, the range of the ratio of the molar weight of the first monomer to the molar weight of the total monomer amount is 0.1-0.8, the range of the ratio of the molar weight of the aminopropyltriethoxysilane in the second monomer to the molar weight of the total monomer amount is 0.1-0.4, and the range of the ratio of the molar weight of the glyoxal in the second monomer to the molar weight of the total monomer amount is 0.1-0.5. The total monomer amount refers to the total amount of the tetraethyl orthosilicate, the aminopropyltriethoxysilane and the glyoxal.

As an example, the ratio of the molar weight of the first monomer to the molar weight of the total monomer amount may be 0.2, 0.3, 0.4, 0.5, 0.6 or 0.7.

As an example, the ratio of the molar weight of the aminopropyltriethoxysilane in the second monomer to the molar weight of the total monomer amount is 0.2, 0.25, 0.3 or 0.35.

As an example, the ratio of the molar weight of the glyoxal in the second monomer to the molar weight of the total monomer amount is 0.15, 0.2, 0.25, 0.3, 0.35, 0.4 or 0.45.

In an exemplary embodiment, the molar weight of the first monomer may be greater than the molar weight of the aminopropyltriethoxysilane in the second monomer.

In an exemplary embodiment, the molar weight of the first monomer may be greater than the molar weight of the glyoxal in the second monomer.

In an exemplary embodiment, the molar weight of the first monomer may be greater than the total molar weight of the aminopropyltriethoxysilane and the glyoxal in the second monomer.

In the case that the first monomer comprises tetraethyl orthosilicate, and the second monomer comprises aminopropyltriethoxysilane and glyoxal, the polycondensate of the first monomer and the second monomer is a polycondensate of tetraethyl orthosilicate, aminopropyltriethoxysilane and glyoxal. Because the second monomer comprises aminopropyltriethoxysilane, the siloxy groups in the formed polycondensate, in hydrolysis, generate rich silicon hydroxyl groups (—Si—OH), and therefore the surface (the modifying layer KX) of the polymer microsphere finally has silicon hydroxyl (—Si—OH).

When the surface (the modifying layer KX) of the polymer microsphere has silicon hydroxyl (—Si—OH), the polymer microsphere may be used for the extraction and the purification of RNA.

In at least one embodiment of the present application, the first monomer comprises styrene, the second monomer comprises ethyleneimine, the range of the ratio of the molar weight of the first monomer to the molar weight of the total monomer amount is 0.1-0.6, and the range of the ratio of the molar weight of the second monomer to the molar weight of the total monomer amount is 0.4-0.9. The total monomer amount refers to the total molar weight of the styrene and the ethyleneimine.

As an example, the ratio of the molar weight of the first monomer to the molar weight of the total monomer amount may be 0.2, 0.3, 0.4 or 0.5.

As an example, the ratio of the molar weight of the second monomer to the molar weight of the total monomer amount may be 0.5, 0.6, 0.7 or 0.8.

As an example, the molar weight of the first monomer may be greater than the molar weight of the second monomer.

In the case that the first monomer comprises styrene, and the second monomer comprises ethyleneimine, the copolymer of the first monomer and the second monomer is a copolymer of styrene and ethyleneimine. Because the copolymer comprises imido, the surface (the modifying layer KX) of the polymer microsphere finally has amino (—NH₂).

When the surface (the modifying layer KX) of the polymer microsphere has amino (—NH₂), the polymer microsphere may be used for the extraction and the purification of antibody proteins.

In at least one embodiment of the present application, as shown in FIG. 3, the range of the distance D from each of the magnetic particles HC to the modifying layer KX in the direction from a geometric center of the polymer microsphere pointing to the modifying layer KX is 10 nm-500 nm.

As an example, the distance D from each of the magnetic particles HC to the modifying layer KX in the direction from a geometric center of the polymer microsphere pointing to the modifying layer KX may be 15 nm, 30 nm, 50 nm, 100 nm, 150 nm, 180 nm, 200 nm, 300 nm, 350 nm, 280 nm, 400 nm, 450 nm or 480 nm.

In at least one embodiment of the present application, the magnetic particles HC comprise one or more of an iron oxide, a cobalt oxide and a nickel oxide, and the range of the size of each of the magnetic particles HC is 8 nm-15 nm.

As an example, the size of each of the magnetic particles may be 9 nm, 10 nm, 11 nm, 12 nm, 13 nm or 14 nm.

As an example, the magnetic particles may comprise a ferrite-type material, whose main component is MO·6Fe₂O₃, wherein M represents Ba, Sr, Pb or a composite component such as SrCa and LaCa.

As an example, the magnetic particles may comprise at least one of ferroferric oxide and ferric oxide.

In the embodiments of the present application, because it is configured that the range of the size of each of the magnetic particles is 8 nm-15 nm, in order to increase the smoothness of the surface of the polymer microsphere, prevent that the surface of the polymer microsphere has unevenness after the shell K has coated the core H, and prevent that the shell K of the polymer microsphere cracks by the effect of an external force, it is configured that the minimum distance D from each of the magnetic particles HC to the modifying layer KX in the direction from a geometric center of the polymer microsphere pointing to the modifying layer KX is close to the size of each of the magnetic particles HC, or, in other words, the minimum distance D from each of the magnetic particles HC to the modifying layer KX in the direction from a geometric center of the polymer microsphere pointing to the modifying layer KX is 10 nm, thereby increasing the smoothness of the surface of the polymer microsphere, and reducing the probability with which by the effect of an external force the shell K of the polymer microsphere cracks. In addition, if the distance D from each of the magnetic particles HC to the modifying layer KX in the direction from a geometric center of the polymer microsphere pointing to the modifying layer KX is greater than 500 nm, the content of the magnetic particles HC in the polymer microsphere decreases, and the effect of magnetic response is deteriorated.

In at least one embodiment of the present application, the range of the size of the polymer microsphere is 100 nm-5 μm.

As an example, the size of the polymer microsphere may be 200 nm, 300 nm, 500 nm, 800 nm, 1 μm, 1.5 μm, 2 μm, 2.5 μm, 3 μm, 3.5 μm, 4 μm or 4.5 μm.

It should be noted that all of the sizes in the description are average values, and all of fluctuations in the range of 3%-5% around the average values fall within the protection scope of the present application.

The preparing method of the polymer microsphere will be described below.

In practical applications, magnetic microspheres are prepared usually by firstly preparing the magnetic particles and subsequently coating the magnetic particles with a polymer or silicon-dioxide layer. The methods for preparing the magnetic particles in the related art include sol-gel process, coprecipitation, hydrothermal method, high-temperature organic-phase thermolysis and so on. The particular processes of those methods may refer to the description in the related art, and are not discussed further herein.

The magnetic microspheres prepared by the preparing methods in the related art, because of the restrictions by the sizes and the thermal stability, have the problem of a poor effect of magnetic response or an unstable magnetic adsorption force in practical applications.

In view of that, an embodiment of the present application provides a preparing method of a polymer microsphere, wherein the preparing method is applied to prepare the polymer microsphere stated above, and the method comprises:

S801: forming the magnetic particles HC.

As an example, the magnetic particles HC comprise one or more of an iron oxide, a cobalt oxide and a nickel oxide, and the range of the size of each of the magnetic particles HC is 8 nm-15 nm.

For example, the magnetic particles HC may include γ-Fe₃O₄ nanoparticles.

S802: mixing the magnetic particles HC, at least one first monomer and at least one second monomer together, to form the polymer microsphere in one step by in-situ polymerization, wherein the polymer microsphere comprises a core H and a shell K coating the core H; the core H comprises at least two magnetic particles HC and a dispersion HF located between two neighboring magnetic particles HC; the shell K comprises a main shell body KB and a modifying layer KX, and the modifying layer KX is located on the side of the main shell body KB that is away from the magnetic particles HC; and all of the dispersion HF, the main shell body KB and the modifying layer KX have the same materials and comprise a polymer formed by at least one first monomer and at least one second monomer.

It should be noted that the step S802 is performed during continuous stirring, to facilitate the magnetic particles HC to be dispersed well. The stirring includes mechanical stirring and sonication, thereby further improving the effect of the dispersion of the magnetic particles HC in the system, to facilitate to obtain the magnetic particles HC separately coated by the dispersion HF.

In an exemplary embodiment, the dispersion HF is used to disperse and isolate the magnetic particles HC, to prevent that, in the situation of thermal cycle, two neighboring magnetic particles HC have interface interfusion therebetween.

In at least one embodiment of the present application, the dispersion HF covers at least part of the surfaces of the magnetic particles HC. That the dispersion HF is configured to be capable of coating at least part of the surfaces of the magnetic particles HC includes but is not limited to the following cases:

In the first case, all of the magnetic particles HC are coated separately by the dispersion HF, in which case the dispersion HF is configured to be capable of coating all of the surfaces of the magnetic particles HC.

In the second case, regarding all of the magnetic particles HC, two or more magnetic particles HC that are coated together by the dispersion HF exist, in which case the dispersion HF is configured to be capable of coating part of the surfaces of the magnetic particles HC.

In the third case, all of the magnetic particles HC include a first part and a second part. The first part of the magnetic particles HC are coated separately by the dispersion HF, and the dispersion HF is configured to be capable of coating all of the surfaces of the first part of the magnetic particles HC. In the second part of the magnetic particles HC, two or more magnetic particles HC are coated together by the dispersion HF, and the dispersion HF is configured to be capable of coating part of the surfaces of the magnetic particles HC.

In an exemplary embodiment, the modifying layer HX comprises functionalizing functional groups, for example, at least one of amino, carboxyl, hydroxyl and silicon hydroxyl, so that the surface of the polymer microsphere has at least one of those functionalizing functional groups.

In the embodiments of the present application, because the material of the dispersion HF, the material of the main shell body KB and the material of the modifying layer KX are the same, the dispersion HF and the main shell body KB also comprise the functionalizing functional groups, for example, at least one of amino, carboxyl, hydroxyl and silicon hydroxyl.

The in-situ polymerization method comprises adding all of the reactive monomers (the first monomer and the second monomer) and the catalyst into a dispersion phase (magnetic particle dispersion liquid) and mixing them together. Because the reactive monomers are soluble in the reaction-solvent system, and their polymer is insoluble in the entire system, the polymerization reaction happens on the magnetic particles HC. After the reaction begins, the reactive monomers prepolymerize, and the prepolymer polymerizes. When the prepolymer polymerizes and the size gradually increases, they are deposited on the surfaces of the magnetic particles HC, further coat the magnetic particles HC, and at the same time the dispersion HF, the main shell body KB and the modifying layer KX of the polymer microsphere are formed.

In the embodiments of the present application, the polymer microsphere is synthesized by means of in-situ polymerization. The core structure H in the polymer microsphere comprises the magnetic particles HC and the dispersion HF, and when the quantity of the magnetic particles HC in the core structure H is two or more, the dispersion HF facilitates to separate the interfaces between the magnetic particles HC, which prevents that, in the case of thermal cycle, the interfaces of the magnetic particles HC in the core structure H interfuse. The shell structure K of the polymer microsphere comprises the main shell body KB and the modifying layer KX, wherein the main shell body KB serves to protect the core structure H, and the modifying layer KX causes the surface of the shell K to have particular functional groups. By configuring that the material of the dispersion HF, the material of the main shell body KB and the material of the modifying layer KX are the same, that can, to a large extent, simplify the configuring of the shell structure K and simplify the preparing process of the polymer microsphere, thereby reducing the cost while ensuring its quality.

In at least one embodiment of the present application, the step S802 of mixing the magnetic particles, the at least one first monomer and the at least one second monomer together, to form the polymer microsphere in one step by in-situ polymerization includes two cases:

In the first case, the preparation is performed by in-situ copolymerization, which comprises: S8021: mixing the magnetic particles HC, one first monomer and one second monomer together, to form the polymer microsphere in one step by in-situ polymerization.

In at least one embodiment of the present application, the first monomer comprises styrene, and the second monomer comprises methacrylic acid. In this case, the step S8021 comprises:

S8021A: dispersing the magnetic particles HC in anhydrous ethanol, to obtain a suspension of the magnetic particles HC.

For example, this step comprises transferring 0.23 g Fe₂O₃ nanoparticles and 40 mL anhydrous ethanol into a four-neck flask, and sonicating the mixture for 5 minutes. In practical applications, the sonication duration may be adjusted properly according to the different powers of the sonicating device, for example, sonicating for 10 minutes or 15 minutes, to facilitate the Fe₂O₃ nanoparticles to be dispersed.

S8021B: adding polyvinylpyrrolidone (PVP), an initiator and the second monomer into the suspension of the magnetic particles HC, and introducing a protective gas; and after stirring uniformly, adding the first monomer, and stirring continuously at a first preset temperature to react for 2-6 hours, wherein the first monomer comprises styrene, and the second monomer comprises methacrylic acid.

The stirring includes mechanical stirring and sonication. Because sonication might sharply generate heat, the continuous stirring to react for 2-6 hours includes two cases of continuous mechanical stirring and alternated performance of mechanical stirring and sonication, and does not include continuous performance of sonication.

The addition of polyvinylpyrrolidone (PVP) facilitates to increase the molecular weight of the polymer. Ethanol itself has a poor solubility to the copolymer, and the addition of polyvinylpyrrolidone (PVP) facilitates the copolymer to be better dissolved into the ethanol solution, whereby the reaction can continue to obtain a higher molecular weight. The effect of the PVP addition in the below preparing methods is similar to that of this place, and is not discussed further.

As an example, the first preset temperature may be 80±5° C., for example, 80±2° C.

As an example, the protective gas may be an inert gas, for example, nitrogen (N₂).

As an example, the initiator may comprise dibenzoyl peroxide (BPO), and the range of the ratio of the initiator to the total molar weight of the reactive monomers is 0.01-0.1. For example, the ratio of the initiator to the total molar weight of the reactive monomers may be 0.02, 0.03, 0.04, 0.05, 0.06, 0.07, 0.08 or 0.09.

As an example, the range of the ratio of the molar weight of the first monomer to the molar weight of the second monomer is 0.1-10. For example, the ratio of the molar weight of the first monomer to the molar weight of the second monomer may be 0.2, 0.3, 0.4, 0.5, 0.6, 0.7, 0.8, 0.9, 1.0, 1.1, 1.5, 1.8, 3, 3.5, 4, 4.5, 5, 5.5, 6, 6.5, 7, 7.5, 8, 8.5, 9 or 9.5.

For example, this step comprises adding 0.25 g polyvinylpyrrolidone (PVP), 0.025 g dibenzoyl peroxide (BPO) and 0.5 mL redistilled methacrylic acid (AA) into the suspension of the magnetic particles HC; stirring uniformly (ultrasonically dispersing uniformly), introducing nitrogen for protection, mechanically stirring the mixture liquid at 30° C. for 1 hours, and adding 2 mL redistilled styrene into the mixture liquid; and continuously mechanically stirring at 78° C. to react for 4 hours, with intermittent sonication during the reaction process.

S8021C: after the reaction liquid has cooled to room temperature, washing by using anhydrous ethanol and deionized water, and drying to obtain the polymer microsphere.

For example, this step comprises, after the reaction liquid has cooled to room temperature, magnetically separating the obtained product, washing by using ethanol and water several times, and drying in a vacuum oven at 40° C. for 12 hours, to obtain a Fe₂O₃/PS-PAA polymer microsphere. The material of the magnetic particles HC is Fe₂O₃. All of the materials of the dispersion HF, the main shell body KB and the modifying layer KX are a copolymer of styrene and methacrylic acid (PS-PAA).

In the case that the first monomer comprises styrene, the second monomer comprises methacrylic acid, and the initiator comprises dibenzoyl peroxide (BPO), their reaction mechanism is shown in FIG. 5 to FIG. 7.

In FIG. 5, the dibenzoyl peroxide (BPO), when heated, has thermolysis, to generate a free radical PHCOO·, and the free radical PHCOO· further reacts to generate a free radical PH· and CO₂.

In FIG. 6, the free radical PH· polymerizes sequentially with the methacrylic acid and the styrene, to obtain a prepolymer

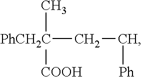

wherein PH represents phenyl.

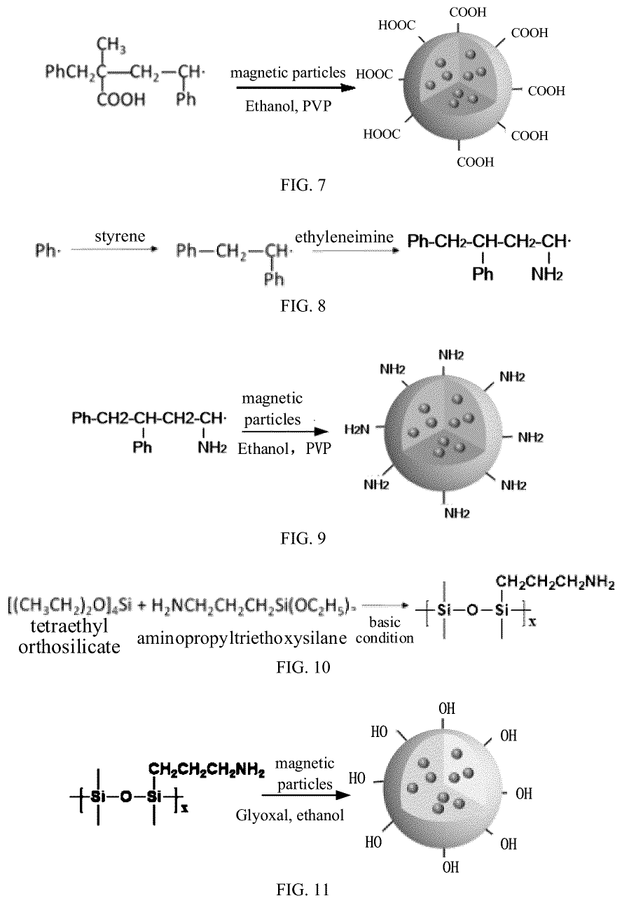

In FIG. 7, the prepolymer deposits on the ferric-oxide nanoparticles and further polymerizes, to simultaneously obtain the dispersion HF, the main shell body KB and the modifying layer KX. At this point, the surface of the polymer microsphere has carboxyl (—COOH).

The structural formula of the copolymer of styrene and methacrylic acid (PS-PAA) is as follows:

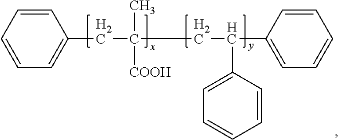

wherein both of x and y are a positive integer.

In at least one embodiment of the present application, the first monomer comprises styrene, and the second monomer comprises ethyleneimine. In this case, the step S8021 comprises:

S8021a: dispersing the magnetic particles HC in anhydrous ethanol, to obtain a suspension of the magnetic particles.

For example, this step comprises transferring 5 g Fe₃O₄ nanoparticles and 10 mL anhydrous ethanol into a four-neck flask, and sonicating the mixture for 5 minutes. In practical applications, the sonication duration may be adjusted properly according to the different powers of the sonicating device, for example, sonicating for 10 minutes or 15 minutes, to facilitate the Fe₃O₄ nanoparticles to be dispersed.

S8021b: adding polyvinylpyrrolidone (PVP), an initiator, the first monomer and the second monomer into the suspension of the magnetic particles HC; and stirring continuously under vacuum at a second preset temperature to react for 2-4 hours, wherein the first monomer comprises styrene, and the second monomer comprises ethyleneimine.

The stirring includes mechanical stirring and sonication. Because sonication might sharply generate heat, the continuous stirring to react for 2-4 hours includes two cases of continuous mechanical stirring and alternated performance of mechanical stirring and sonication, and does not include continuous performance of sonication.

As an example, the second preset temperature may be 130±10° C., for example, 130±5° C. or 130±2° C.

As an example, the free-radical initiator may comprise dibenzoyl peroxide (BPO), and the range of the ratio of the free-radical initiator to the total molar weight of the reactive monomers is 0.01-0.1. For example, the ratio of the free-radical initiator to the total molar weight of the reactive monomers may be 0.02, 0.03, 0.04, 0.05, 0.06, 0.07, 0.08 or 0.09.

As an example, the range of the ratio of the molar weight of the first monomer to the molar weight of the total monomer amount is 0.1-0.6, and the range of the ratio of the molar weight of the second monomer to the molar weight of the total monomer amount is 0.4-0.9. The total monomer amount refers to the total molar weight of the styrene and the ethyleneimine.

For example, this step comprises adding 0.25 g polyvinylpyrrolidone (PVP), 0.025 g dibenzoyl peroxide (BPO), 5.4 g ethyleneimine and 15 mL redistilled styrene into the suspension of the magnetic particles HC; and vacuumizing to 2 bar, heating to 135±1° C., and mechanically stirring to react for 3 hours.

S8021c: after the reaction liquid has cooled to room temperature, washing by using anhydrous ethanol and deionized water, and drying to obtain the polymer microsphere.

This step comprises, after the reaction ends and cools to room temperature, transferring the reaction liquid into a plastic container, placing onto a magnetic-force support, removing the supernatant, washing by using 200 mL deionized water 2 times, till the pH value of the washing liquid becomes approximately 7, washing by using anhydrous ethanol 2 times, and drying, to obtain a Fe₃O₄/PS-PEI polymer microsphere. The material of the magnetic particles HC is Fe₃O₄. All of the materials of the dispersion HF, the main shell body KB and the modifying layer KX are a copolymer of styrene and ethyleneimine (PS-PEI).

In the case that the first monomer comprises styrene, the second monomer comprises ethyleneimine, and the initiator comprises dibenzoyl peroxide (BPO), their reaction mechanism is shown in FIG. 5, FIG. 8 and FIG. 9.

In FIG. 5, the dibenzoyl peroxide (BPO), when heated, has thermolysis, to generate a free radical PHCOO·, and the free radical PHCOO· further reacts to generate a free radical PH· and CO₂.

In FIG. 8, the free radical PH· polymerizes sequentially with the styrene and the ethyleneimine, to generate a prepolymer

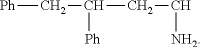 [SMILES: NCCC(Cc1ccccc1)c1ccccc1]

In FIG. 9, the prepolymer deposits on the ferric-oxide nanoparticles and further polymerizes, to simultaneously obtain the dispersion HF, the main shell body KB and the modifying layer KX. At this point, the surface of the polymer microsphere has amino (—NH₂).

The structural formula of the copolymer of styrene and ethyleneimine (PS-PEI) is as follows:

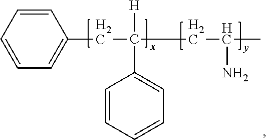

wherein both of x and y are a positive integer.

The above-described preparing mode of in-situ copolymerization according to the embodiments of the present application can reduce the processes, and reduce the consumption of the raw materials, thereby further reducing the cost, and, by controlling the ratio of the molar concentrations of the main-body monomer (the first monomer) and the functionalizing monomer (the second monomer) in the copolymer, can adjust the contents of the surface groups of the polymer microsphere, thereby alleviating the problem of agglomeration between the polymer microspheres caused by a too high group density.

In the second case, the preparation is performed by in-situ condensation polymerization, which comprises:

S8022: mixing the magnetic particles, one instance of the first monomer and two instances of the second monomer together, to form the polymer microsphere in one step by in-situ polymerization.

In at least one embodiment of the present application, the first monomer comprises tetraethyl orthosilicate, and the second monomer comprises aminopropyltriethoxysilane and glyoxal. In this case, the step S8022 comprises:

S8022A: dispersing the first monomer in anhydrous ethanol.

For example, this step comprises adding 15 mL tetraethyl orthosilicate into a reaction bulb comprising 50 mL anhydrous ethanol, and magnetically stirring for 15 minutes.

S8022B: dispersing the magnetic particles HC in a water solution comprising polyvinylpyrrolidone (PVP), to obtain a suspension of the magnetic particles HC.

For example, this step comprises dispersing 22.5 g ferroferric-oxide nanoparticles into a 50 mL polyvinylpyrrolidone (PVP) solution, and stirring (magnetically stirring, mechanically stirring or ultrasonically dispersing) for 15 minutes.

S8022C: adding the suspension of the magnetic particles HC into the anhydrous ethanol in which the first monomer is dispersed, subsequently adding the two second monomers and ammonia, stirring uniformly, and subsequently reacting at a third preset temperature for 1-3 hours, wherein the first monomer comprises tetraethyl orthosilicate, and the second monomer comprises aminopropyltriethoxysilane and glyoxal.

As an example, the third preset temperature may be 80±5° C., for example, 80±2° C.

The range of the ratio of the molar weight of the first monomer to the molar weight of the total monomer amount is 0.1-0.8, the range of the ratio of the molar weight of the aminopropyltriethoxysilane in the second monomer to the molar weight of the total monomer amount is 0.1-0.4, and the range of the ratio of the molar weight of the glyoxal in the second monomer to the molar weight of the total monomer amount is 0.1-0.5. The total monomer amount refers to the total amount of the tetraethyl orthosilicate, the aminopropyltriethoxysilane and the glyoxal.

For example, this step comprises adding a ferroferric-oxide dispersion liquid into a solution of tetraethyl orthosilicate, adding 15 mL aminopropyltriethoxysilane, 15 mL hexandial and 50 mL ammonia with a concentration of 5% into the mixture liquid, stirring sufficiently for 10 minutes, heating the reaction liquid to 80° C., and continuously reacting for 2 hours.

In the case that the first monomer comprises tetraethyl orthosilicate, and the second monomer comprises aminopropyltriethoxysilane and glyoxal, the polycondensate of the first monomer and the second monomer is a polycondensate of tetraethyl orthosilicate, aminopropyltriethoxysilane and glyoxal. Because the second monomer comprises aminopropyltriethoxysilane, the siloxy groups in the formed polycondensate, in hydrolysis, generate rich silicon hydroxyl groups (—Si—OH), and therefore the surface (the modifying layer KX) of the polymer microsphere finally has silicon hydroxyl (—Si—OH).

S8022D: after the reaction liquid has cooled to room temperature, washing by using anhydrous ethanol and deionized water, and drying to obtain the polymer microsphere.

For example, this step comprises, after the reaction ends and cools to room temperature, transferring the reaction liquid into a plastic container, placing onto a magnetic-force support, removing the supernatant, washing by using 200 mL deionized water at least 2 times, till the pH value of the washing liquid becomes approximately 7, washing by using anhydrous ethanol 2 times, and drying, to obtain a polymer microsphere whose surface has silicon hydroxyl.

In the case that the first monomer comprises tetraethyl orthosilicate, and the second monomer comprises aminopropyltriethoxysilane and glyoxal, their reaction mechanism is shown in FIG. 10 to FIG. 12.

As shown in FIG. 10, the tetraethyl orthosilicate and the aminopropyltriethoxysilane have condensation polymerization in a basic condition (ammonia), to obtain an intermediate product

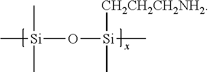

As shown in FIG. 11, the intermediate product deposits on the ferric-oxide nanoparticles and further polymerizes, to simultaneously obtain the dispersion HF, the main shell body KB and the modifying layer KX. Because the second monomer comprises aminopropyltriethoxysilane, the siloxy groups in the formed polycondensate, in hydrolysis, generate rich silicon hydroxyl groups (—Si—OH), and therefore the surface (the modifying layer KX) of the polymer microsphere finally has silicon hydroxyl (—Si—OH). The figures according to the embodiments of the present application merely illustrate the hydroxyl group (—OH) on the surface of the polymer microsphere, and do not illustrate the silicon atom (Si).

The structural formula of the polycondensate of tetraethyl orthosilicate, aminopropyltriethoxysilane and glyoxal is as follows:

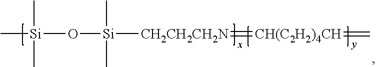

wherein both of x and y are a positive integer.

The above-described preparing mode of in-situ condensation polymerization according to the embodiments of the present application can reduce the processes, and reduce the consumption of the raw materials, thereby further reducing the cost, and, by controlling the ratio of the molar concentrations of the main-body monomer (the first monomer) and the functionalizing monomer (the second monomer) in the polycondensate, can adjust the contents of the surface groups of the polymer microsphere, thereby alleviating the problem of agglomeration between the polymer microspheres caused by a too high group density.

In the embodiments of the present application, the step of preparing the magnetic particles HC includes the following two methods:

The first method of preparing the magnetic particles HC is as follows:

Step S801A: mixing ferric trichloride hydrate, ferrous chloride hydrate and isopropanol together, and stirring uniformly.

For example, the particular process may comprise: adding 0.5 g FeCl₃·6H₂O, 50 mL nitrogen-purified HPLC-grade isopropanol and a magnetic stir bar into a 250 mL two-neck flask; and subsequently adding 0.25 g FeCl₂·4H₂O, and stirring sufficiently, wherein after complete dissolving, the color of the solution is yellowish orange. The isopropanol is the reaction solvent.

Step S801B: heating to 50° C.±5° C., dripping concentrated ammonia slowly, and stirring to react for 10-30 minutes.

For example, the particular process may comprise: heating the reaction liquid to 50° C., and dripping slowly excessive concentrate ammonia (<10 mL) into the reaction liquid; and reacting for 15 min, when the color of the solution becomes dark brown, and continuously stirring till it is cooled to room temperature.

Step S801C: after the reaction ends, standing the reaction liquid in an environment of 0-10° C. for 4-6 hours.

For example, this step comprises standing in an environment of 0-10° C. for 5 hours, so that the Fe₂O₃ magnetic particles precipitate.

Step S801D: washing by using methanol, adding a methanol solution dissolving oleic acid, and stirring for 2-5 hours, to obtain the magnetic particles.

This step comprises subsequently washing the precipitated black solid by using methanol 3 times (to wash off the residual solvent), and subsequently centrifuging at 10000 rpm for 5 minutes to precipitate the solid; and dissolving 10 mMol oleic acid into 50 mL methanol, adding into the solid, and stirring for 3 hours. The oleic acid can coat the surface of the ferric oxide (Fe₂O₃) crystal to serve for protection, and the methanol can remove excessive oleic acid.

Step S801E: re-washing by using methanol, and dispersing the obtained magnetic particles into hexane.

This step comprises washing by using methanol multiple times, and finally dispersing the product into 50 mL hexane. The hexane serves as the solution dispersant, and can effectively disperse the ferric oxide coated by the oleic acid, and at the same time further protect the ferric oxide crystal.

The above-described method obtains an oleic-acid-stabilized Fe₂O₃ magnetic-particle suspension, wherein the Fe₂O₃ magnetic particles are of a single-crystal structure, and the range of the average diameter is 5-15 nm. After drying, the reaction yield is approximately 86%.

The reaction equations involved in the first preparing method of the magnetic particles HC are as follows:

FeCl₃+FeCl₂+5NH₄OH→Fe(OH)₃+Fe(OH)₂+5NH₄Cl

Fe(OH)₃+Fe(OH)₂+OH⁻→Fe₂O₃+3H₂O(the pH value of the reaction liquid is controlled to be 9-10)

The magnetic particles HC obtained by using the first preparing method are Fe₂O₃.

The second preparing method of the magnetic particles HC is as follows:

S801a: mixing ferric trichloride, ferrous chloride, hydrochloric acid and deionized water together, and stirring uniformly, to obtain an iron-ion mixture liquid.

For example, the particular process may comprise: placing 3.24 g ferric trichloride and 2 g ferrous chloride tetrahydrate into a 200 ml conical flask, adding 50 mL deionized water and 6 mL 1 Mol hydrochloric acid solution (which facilitates the ferric trichloride and the ferrous chloride to be stably dispersed), stirring to dissolve, transferring into a volumetric flask, and filling by using deionized water to 60 mL, to obtain an iron-ion mixture liquid.

S801b: introducing a protective gas, dripping the iron-ion mixture liquid into a sodium-hydroxide solution at 80° C.±5° C. under continuous stirring, and after the iron-ion mixture liquid is dripped completely, reacting for 20-40 minutes.

For example, the particular process may comprise: taking a 250 mL two-neck round-bottom reaction bulb, a magnetic stir bar, a constant-pressure dropping funnel and a breather plug, adding the magnetic stir bar and 100 mL deoxidized 2 Mol sodium-hydroxide solution into the reaction bulb, stirring uniformly, placing the constant-pressure dropping funnel onto a vertical bottle opening of the reaction bulb, and introducing nitrogen for protection; and adding 60 mL of the iron-ion mixture liquid into the constant-pressure dropping funnel by using a syringe, reacting with the nitrogen protection, heating the sodium-hydroxide solution to 80° C., dripping the iron-ion mixture liquid into the sodium-hydroxide solution within 10 minutes for reaction, after the dripping has completed, continuing the reaction for 30 minutes, and, after the reaction ends, cooling to room temperature.

S801c: after the reaction ends, cooling to room temperature, washing by using anhydrous ethanol and deionized water, and drying to obtain the magnetic particles.

For example, the particular process may comprise: transferring the reaction liquid into a plastic container, placing onto a magnetic-force support, and removing the supernatant; subsequently washing by using 200 mL deionized water 5 times, till the pH value of the washing liquid becomes approximately 7; and washing by using anhydrous ethanol, drying, and weighing to obtain that the reaction yield is 91%.

The reaction equation involved in the second preparing method of the magnetic particles HC is as follows:

2Fe³⁺+Fe²⁺+8OH⁻→Fe₃O₄+4H₂O

The magnetic particles HC obtained by using the second preparing method are Fe₃O₄.

In order to test the performance of the polymer microsphere according to the embodiments of the present application, the effects of different types of the polymer microsphere in nucleic acid extraction processes will be described below.

Firstly, the comparison of the performances of different polymer microspheres (also referred to as magnetic beads) in the DNA extraction process will be described:

Various biological reagents will be involved below. Firstly, the components of the biological reagent involved in the DNA extraction will be described.

The DNA extraction reagent system comprises:
1: an erythrocyte lysing liquid, whose components are 5 mMol NaCl, 5% Tween 20, 320 mMol glucose and a 10 mMol trometamol (Tris) solution, wherein the pH of the erythrocyte lysing liquid is approximately 8, for example, pH=8.2.

2: a lysing liquid for lysing the other cells other than erythrocyte, whose components are 3.5 Mol guanidinium isothiocyanate (GITC), 0.1% Tween 20, a 100 mMol TE buffer solution (pH 7.5), 10 mMol ethylenediamine tetraacetic Acid (EDTA), 0.5% sodium dodecyl sulfonate, and 1% polyethylene glycol octylphenol ether (Triton X-100), wherein the TE buffer solution is prepared from Tris and EDTA, is mainly used to dissolve nucleic acids, and can stably store DNA and RNA.

3: protease K (Proteinase K) with a concentration of 10 mg/mL, a 50 mMol trometamol (Tris) solution (the pH is approximately 8), 5 mMol CaCl₂), and 20 vol % glycerin (also referred to as glycerol).

4: a binding fluid for facilitating the polymer microsphere and the nucleic acid to bind together, whose components are:

0.5 Mol NaCl, 45% isopropanol, 8-15% polyethylene glycol (PEG8000, whose average molecular weight is approximately 8000), a 100 mMol trometamol (Tris) solution (the pH is approximately 8.0), and 5 mMol ethylenediamine tetraacetic acid (EDTA).

5: a first washing liquid, whose components are a 1× TE buffer solution and a 75% ethanol solution.

6: a second washing liquid, whose components are a 1× TE buffer solution and an 80% ethanol solution, wherein both of the two washing liquids are used to wash proteins and salts, and the first washing liquid has a lower ethanol concentration, and has a solubility to the nucleic acid, thereby preventing removing the nucleic acid, and reducing the extraction rate of the nucleic acid.

The 1× TE buffer solution represents the buffer solution of an original concentration.

7: an eluent for eluting the nucleic acid from the polymer microsphere (magnetic bead), whose components are 10 mMol trihydroxymethyl aminomethane (Tris-HCl) and 0.1 mMol ethylenediamine tetraacetic acid (EDTA), i.e., a TE buffer solution.

The DNA extraction process comprises two parts, wherein in the first part the erythrocyte is removed, and in the second part the DNA is extracted.

The particular process of removing the erythrocyte in the first part is as follows:
1: placing 250 microliters of whole blood (bovine whole blood) into a 2 mL centrifuge tube, adding 800 microliters of the erythrocyte lysing liquid, and vibrating sufficiently to mix uniformly for 10 seconds, to obtain a red transparent clear mixture;
2: centrifuging the mixture at a centrifuging rotational speed of 12000 rpm for 1 minute, and removing the supernatant, to obtain a white precipitate;
3: adding again 800 microliters of the erythrocyte lysing liquid into the white precipitate, and vibrating sufficiently to mix uniformly, to suspend the precipitate to obtain a mixture liquid; and
4: centrifuging the mixture liquid at a centrifuging rotational speed of approximately 12000 rpm for 1 minute, and removing the supernatant, to obtain a white precipitate.

The particular process of extracting the DNA in the second part is as follows:
1: adding 400 μL of the lysing liquid into the white precipitate obtained by the removal of the erythrocyte, mixing uniformly with vortex, and reacting at 65° C. for 30 minutes;
2: adding 400 μL isopropanol, and mixing uniformly with vortex;
3: adding 20 μL of the polymer microsphere (also referred to as magnetic bead), for example, the polymer microsphere whose surface has silicon hydroxyl (—Si—OH), the polymer microsphere whose surface has carboxyl (—COOH), or the polymer microsphere whose surface has amino (—NH₂), mixing uniformly with vortex, standing for 5 min, and, every 2 minutes, vortex-treating for 3 seconds;
4: adding 600 μL of the binding fluid, vibrating to mix uniformly with vortex, standing for 10 min, and, every 2 min, vortex-treating for 3 s;
5: centrifuging at a low speed, standing on a magnetic-force support for 2 min, and removing the supernatant;
6: adding 600 μL of the first washing liquid into the product of the 5th step, and mixing uniformly by blowing and sucking (sucking up and blowing down with a pipette, thereby mixing uniformly);
7: standing on a magnetic-force support to adsorb for 2 min, and removing the supernatant;
8: adding 600 μL of the second washing liquid into the product of the 7th step, and mixing uniformly by blowing and sucking;
9: standing on a magnetic-force support to adsorb for 2 min, and air-drying at room temperature till the surface has no obvious liquid;
10: adding 100 μL of non-enzyme water or the TE buffer solution, and vibrating and eluting at room temperature for 10 min; and
11: standing the centrifuge tube of the 10th step on a magnetic-force support for 2 min, and transferring the supernatant into a new centrifuge tube for the next step of the test, wherein the supernatant is the DNA solution.

The DNAs that are obtained by the extraction by using the polymer microsphere whose surface has silicon hydroxyl (—Si—OH), the polymer microsphere whose surface has carboxyl (—COOH), or the polymer microsphere whose surface has amino (—NH₂) undergo tests with respect to the concentrations and the purities, and the data in Table 1 are obtained.

TABLE 1
comparison of the performances in the DNA extraction of the
polymer microspheres according to the present application

| | DNA recovery | DNA purity |
|---|---|---|
| Type of magnetic bead | concentration (ng/μL) | (A260/A280) |
| —COOH | 98/106/102 | 1.78/1.75/1.77 |
| —Si—OH | 78/88/94 | 1.67/1.66/1.69 |
| —NH₂ | 65/63/69 | 1.70/1.72/1.69 |
| Reference group | 93/104/97 | 1.68/1.71/1.64 |

The reference group is a polymer microsphere (magnetic bead) in the related art. It can be seen that the DNA recovery concentration of the polymer microsphere whose surface has carboxyl (—COOH) according to the embodiments of the present application is comparable to that of the magnetic bead in the related art, and the purity of the DNA obtained by the extraction is obviously greater than that of the magnetic bead in the related art. It should be noted that each of the groups of the data in Table 1 includes 3 data, and the 3 data are the results of multiple times of measurement of the same one sample.

The comparison of the performances of different polymer microspheres (also referred to as magnetic beads) in the RNA extraction process will be described below:

Various biological reagents will be involved below. Firstly, the components of the biological reagent involved in the RNA extraction will be described.

The RNA extraction reagent system comprises:
1: an erythrocyte lysing liquid, whose components are the same as those of the erythrocyte lysing liquid of the above-described DNA extraction reagent system, and may particularly refer to the preceding context.
2: a lysing liquid: 4 Mol guanidinium isothiocyanate (GITC), 1 mMol trihydroxymethyl aminomethane (Tris-HCl), and 0.05% Tween 20, with a pH 7.5.
3: a binding fluid: 10% polyethylene glycol (PEG8000, whose average molecular weight is approximately 8000), 1 Mol NaCl, 20 mMol trihydroxymethyl aminomethane (Tris-HCl), 10 mMol ethylenediamine tetraacetic acid (EDTA), and 0.05% Tween 20, with a pH 8.
4: isopropanol.
5: a third washing liquid: 0.1 Mol guanidinium isothiocyanate (GITC), 1 mMol trihydroxymethyl aminomethane (Tris-HCl), 0.05% Tween 20, and 1 Mol NaCl, with a pH 7.5.
6: a fourth washing liquid: an 80% ethanol solution.
7: an eluent: non-enzyme water or a TE buffer solution.
8: 10× Dnase I buffer solution: 100 mMol Tris-HCl, 0.5% Tween 20, 25 mMol MgCl2, and 5 Mmol CaCl₂).

9: a reaction buffer solution: a 1× Dnase I buffer solution, a DNase I buffer solution 20 U/mL, RNasin (RNA enzyme inhibitor) 0.05%, and non-enzyme water.

The RNA extraction process comprises two parts, wherein in the first part the erythrocyte is removed, and in the second part the RNA is extracted.

The removal of the erythrocyte in the first part is similar to the process of the removal of the erythrocyte (the lysing of the erythrocyte) in the preceding context. The particular process of the second part is as follows:
1: adding 500 μL of the lysing liquid and 10 μL protease K into the white precipitate obtained by the removal of the erythrocyte, and vibrating to mix uniformly with vortex at room temperature for 10 seconds, standing at room temperature for 10 minutes, and, every 3 minutes, inverting repeatedly 8-10 times;
2: centrifuging the centrifuge tube of the 1st step at a rotational speed of 12000 g at 4° C. for 2 minutes, and transferring the supernatant into a new centrifuge tube;
3: adding 270 μL of the binding fluid, and mixing uniformly with vortex for 3 seconds;
4: adding 20 μL of the magnetic-bead suspension, and mixing uniformly with vortex for 3 seconds;
5: adding 100 μL isopropanol, mixing uniformly by blowing and sucking with a pipette 8-10 times, standing for 10 minutes, and, every three minutes, inverting repeatedly 8-10 times;
6: centrifuging at a low speed, standing on a magnetic-force support for 2 minutes, and removing the supernatant;
7: adding 300 μL of the third washing liquid, and mixing uniformly by blowing and sucking with a pipette 8-10 times;
8: centrifuging at a low speed, standing on a magnetic-force support for 2 minutes, and removing the supernatant (if DNA enzyme lysis is required, skipping to the 15th step for the operation);
9: adding 700 μL of the second washing liquid, and mixing uniformly by blowing and sucking;
10: standing on a magnetic-force support to adsorb for 2 minutes, and removing the supernatant;
11: repeating the operations of the 9th step and the 10th step one time;
12: air-drying at room temperature for 5 minutes, till the surface has no obvious liquid;
13: adding 40 μL of the eluent, mixing uniformly sufficiently by blowing and sucking, and vibrating at 1300 rpm at room temperature for 5 minutes;
14: standing the centrifuge tube on a magnetic-force support for 2 minutes, and transferring the supernatant into a new centrifuge tube for the next step of the test;
if DNA enzyme lysis is required, skipping from the 8th step to the 15th step;
15: adding 100 μL of the reaction buffer solution into the centrifuge tube of the 8th step, mixing uniformly by blowing and sucking, and reacting and incubating at room temperature for 15 minutes;
16: adding 500 μL of the binding fluid, mixing uniformly sufficiently by blowing and sucking, standing on ice for 5 minutes, and, every two minutes, inverting 10 times;
17: standing on a magnetic-force support to adsorb for 2 minutes, and removing the supernatant;
18: repeating the operations of the 9th step and the 10th step twice;
19: air-drying at room temperature for 5 minutes, till the surface has no obvious liquid;
20: adding 60 μL-100 μL of the eluent, mixing uniformly sufficiently by blowing and sucking, and vibrating at 1300 rpm at room temperature for 5 minutes; and
21: standing the centrifuge tube on a magnetic-force support for 2 minutes, and transferring the supernatant into a new centrifuge tube for the next step of the test.

The RNAs that are obtained by the extraction by using the polymer microsphere whose surface has silicon hydroxyl (—Si—OH), the polymer microsphere whose surface has carboxyl (—COOH), and the polymer microsphere whose surface has amino (—NH₂) undergo tests with respect to the concentrations and the purities, and the data in Table 2 are obtained.

TABLE 2
comparison of the performances in the RNA extraction of the
polymer microspheres according to the present application

| | RNA recovery | RNA purity | |
|---|---|---|---|
| Type of magnetic bead | concentration (ng/μL) | (A260/A280) | RIN |
| —COOH | 16/23/19 | 1.92/1.89/2.05 | 8.9 |
| —Si—OH | 37/32/35 | 1.97/2.01/2.03 | 9.6 |
| —NH₂ | 22/18/19 | 1.85/1.90/1.79 | 9.2 |
| Reference group | 27/25/28 | 1.98/2.08/1.91 | 9.8 |

The reference group is a polymer microsphere (magnetic bead) in the related art. It can be seen that the recovery concentrations of the RNAs extracted by using the polymer microsphere whose surface has silicon hydroxyl (—Si—OH) and the polymer microsphere whose surface has amino (—NH₂) are comparable to that of the magnetic bead in the related art. Especially, regarding the polymer microsphere whose surface has silicon hydroxyl (—Si—OH), its RNA recovery concentration is greater than the RNA recovery concentration in the related art, and its RNA purity and RIN value are also higher. It should be noted that each of the groups of the data in Table 2 includes 3 data, and the 3 data are the results of multiple times of measurement of the same one sample.

The index of RNA integrity value (RIN) refers to, based on an overall electrophoretogram, classifying the total RNA masses of eucaryote, and setting the range of the RIN numerical values to be 1-10, wherein 1 represents the most serious degradation, and 10 represents the most intact.

In addition, in order to test the change of the magnetic responsiveness after heating of the polymer microsphere whose surface has silicon hydroxyl (—Si—OH), the polymer microsphere whose surface has carboxyl (—COOH), or the polymer microsphere whose surface has amino (—NH₂) according to the embodiments of the present application, the magnetic bead of the reference group and the three types of magnetic beads according to the present application are heated at 75° C. at the same time for 15 min, and the states of the magnetic attraction force and the magnetic responsiveness of them after being placed on a magnetic-force support for 1 min, 2 min and 5 min are compared.

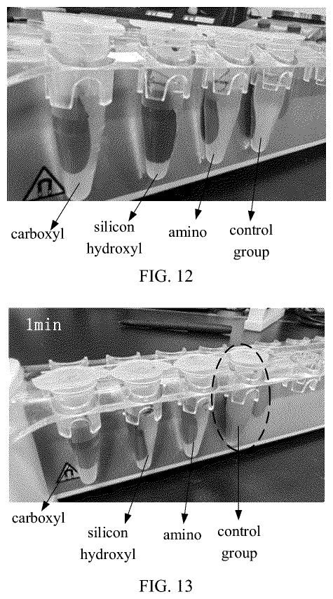

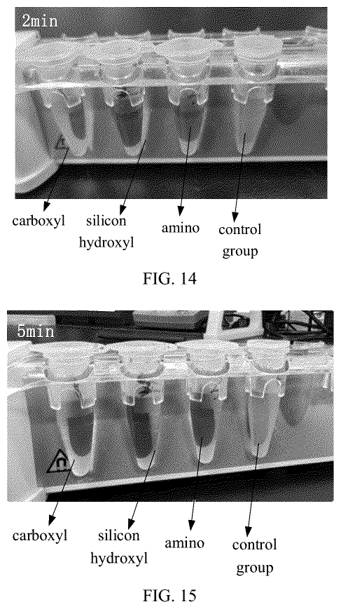

FIG. 12 provides the states of the magnetic attraction force and the magnetic responsiveness of the magnetic beads before the heating. FIG. 13 provides the states after heating at 75° C. for 15 min and standing on a magnetic-force support for 1 min. FIG. 14 provides the states after heating at 75° C. for 15 min and standing on a magnetic-force support for 2 min. FIG. 15 provides the states after heating at 75° C. for 15 min and standing on a magnetic-force support for 5 min. By comparing FIG. 12 to FIG. 15, it can be seen that all of the three types of magnetic beads according to the embodiments of the present application, after being heated at 75° C. for 15 min and standing on a magnetic-force support for 1 min, have good magnetic attraction force and magnetic responsiveness. The magnetic bead of the reference group, after being heated at 75° C. for 15 min and standing on a magnetic-force support for 1 min, is still in the state of a mixed solution, and has poor magnetic attraction force and magnetic responsiveness, and the magnetic bead of the reference group can precipitate out merely after being heated at 75° C. for 15 min and standing on a magnetic-force support for 5 min. Obviously, as compared with the magnetic bead of the reference group (in the related art), all of the three types of magnetic beads according to the embodiments of the present application have a better thermal magnetic stability, which indicates that the magnetic particles HC in the magnetic beads (polymer microspheres) according to the embodiments of the present application are dispersed in the dispersion HF better, and, when heated, the interfaces of the magnetic particles are difficult to interfuse.

The above are merely particular embodiments of the present application, and the protection scope of the present application is not limited thereto. All of the variations or substitutions that a person skilled in the art can easily envisage within the technical scope disclosed by the present application should fall within the protection scope of the present application. Therefore, the protection scope of the present application should be subject to the protection scope of the claims.

1. A polymer microsphere, wherein the polymer microsphere comprises a core and a shell coating the core;
the core comprises at least two magnetic particles and a dispersion located between two neighboring magnetic particles;
the shell comprises a main shell body and a modifying layer, and the modifying layer is located on one side of the main shell body that is away from the magnetic particles; and
a material of the dispersion, a material of the main shell body and a material of the modifying layer are the same.

2. The polymer microsphere according to claim 1, wherein the dispersion covers at least part of a surface of the magnetic particles.

3. The polymer microsphere according to claim 2, wherein each of the material of the dispersion, the material of the main shell body and the material of the modifying layer comprises a polymer formed by at least one first monomer and at least one second monomer; and
a range of a ratio of a molar weight of the first monomer to a molar weight of the second monomer is 0.1-10.

4. The polymer microsphere according to claim 3, wherein each of the second monomer and the modifying layer comprises at least one of amino, carboxyl, hydroxyl and silicon hydroxyl.

5. The polymer microsphere according to claim 4, wherein the molar weight of the second monomer is less than the molar weight of the first monomer.

6. The polymer microsphere according to claim 3, wherein the first monomer comprises at least one of styrene, butadiene, pentadiene, isoprene, methyl propenyl ketone, acrylonitrile, methyl methacrylate and tetraethyl orthosilicate; and
the second monomer comprises at least one of methacrylic acid, ethyleneimine, hydroxyethylene propylene, ethyl orthosilicate, vinyl siloxane, N-(4-carboxylphenyl)maleimide, aminopropyltriethoxysilane and glyoxal.

7. The polymer microsphere according to claim 3, wherein a range of a ratio of a mass of the magnetic particles to a sum of a mass of the first monomer and a mass of the second monomer is 0.5-3.

8. The polymer microsphere according to claim 3, wherein the first monomer comprises styrene, the second monomer comprises methacrylic acid, and the range of the ratio of the molar weight of the first monomer to the molar weight of the second monomer is 0.1-10.

9. The polymer microsphere according to claim 3, wherein the first monomer comprises tetraethyl orthosilicate, the second monomer comprises aminopropyltriethoxysilane and glyoxal, a range of a ratio of the molar weight of the first monomer to a molar weight of a total monomer amount is 0.1-0.8, a range of a ratio of a molar weight of the aminopropyltriethoxysilane in the second monomer to the molar weight of the total monomer amount is 0.1-0.4, and a range of a ratio of a molar weight of the glyoxal in the second monomer to the molar weight of the total monomer amount is 0.1-0.5.

10. The polymer microsphere according to claim 3, wherein the first monomer comprises styrene, the second monomer comprises ethyleneimine, a range of a ratio of the molar weight of the first monomer to a molar weight of a total monomer amount is 0.1-0.6, and a range of a ratio of the molar weight of the second monomer to the molar weight of the total monomer amount is 0.4-0.9.

11. The polymer microsphere according to claim 1, wherein a range of a distance from each of the magnetic particles to the modifying layer in a direction from a geometric center of the polymer microsphere pointing to the modifying layer is 10 nm-500 nm.

12. The polymer microsphere according to claim 1, wherein the magnetic particles comprise one or more of an iron oxide, a cobalt oxide and a nickel oxide, and a range of a size of each of the magnetic particles is 8 nm-15 nm.

13. The polymer microsphere according to claim 1, wherein a range of a size of the polymer microsphere is 100 nm-5 μm.

14. A preparing method of a polymer microsphere, wherein the preparing method is applied to prepare the polymer microsphere according to claim 1, and the method comprises:
forming the magnetic particles; and
mixing the magnetic particles, at least one first monomer and at least one second monomer together, to form the polymer microsphere in one step by in-situ polymerization, wherein the polymer microsphere comprises the core and the shell coating the core; the core comprises at least two magnetic particles and a dispersion located between two neighboring magnetic particles; the shell comprises a main shell body and a modifying layer, and the modifying layer is located on one side of the main shell body that is away from the magnetic particles; and all of the dispersion, the main shell body and the modifying layer have the same materials and comprise a polymer formed by the at least one first monomer and the at least one second monomer.

15. The preparing method according to claim 14, wherein the step of mixing the magnetic particles, the at least one first monomer and the at least one second monomer together, to form the polymer microsphere in one step by in-situ polymerization comprises:
mixing the magnetic particles, one instance of the first monomer and one instance of the second monomer together, to form the polymer microsphere in one step by in-situ polymerization; or
mixing the magnetic particles, one instance of the first monomer and two instances of the second monomer together, to form the polymer microsphere in one step by in-situ polymerization.

16. The preparing method according to claim 15, wherein
the step of mixing the magnetic particles, the one instance of the first monomer and the one instance of the second monomer together, to form the polymer microsphere in one step by in-situ polymerization comprises:
dispersing the magnetic particles in anhydrous ethanol, to obtain a suspension of the magnetic particles;
adding polyvinylpyrrolidone, an initiator and the second monomer into the suspension of the magnetic particles, and introducing a protective gas; and after stirring uniformly, adding the first monomer, and stirring continuously at a first preset temperature to react for 2-6 hours, wherein the first monomer comprises styrene, and the second monomer comprises methacrylic acid; and
after reaction liquid has cooled to room temperature, washing by using anhydrous ethanol and deionized water, and drying to obtain the polymer microsphere.

17. The preparing method according to claim 15, wherein
the step of mixing the magnetic particles, the one instance of the first monomer and the one instance of the second monomer together, to form the polymer microsphere in one step by in-situ polymerization comprises:
dispersing the magnetic particles in anhydrous ethanol, to obtain a suspension of the magnetic particles;
adding polyvinylpyrrolidone, an initiator, the first monomer and the second monomer into the suspension of the magnetic particles; and stirring continuously under vacuum at a second preset temperature to react for 2-4 hours, wherein the first monomer comprises styrene, and the second monomer comprises ethyleneimine; and
after reaction liquid has cooled to room temperature, washing by using anhydrous ethanol and deionized water, and drying to obtain the polymer microsphere.

18. The preparing method according to claim 15, wherein
the step of mixing the magnetic particles, the one instance of the first monomer and the two instances of the second monomer together, to form the polymer microsphere in one step by in-situ polymerization comprises:
dispersing the first monomer in anhydrous ethanol;
dispersing the magnetic particles in a water solution comprising polyvinylpyrrolidone, to obtain a suspension of the magnetic particles;
adding the suspension of the magnetic particles into the anhydrous ethanol in which the first monomer is dispersed, subsequently adding the two second monomers and ammonia, stirring uniformly, and subsequently reacting at a third preset temperature for 1-3 hours, wherein the first monomer comprises tetraethyl orthosilicate, and the second monomer comprises aminopropyltriethoxysilane and glyoxal; and
after reaction liquid has cooled to room temperature, washing by using anhydrous ethanol and deionized water, and drying to obtain the polymer microsphere.

19. The preparing method according to claim 14, wherein the step of forming the magnetic particles comprises:
mixing ferric trichloride hydrate, ferrous chloride hydrate and isopropanol together, and stirring uniformly;
heating to 50° C.±5° C., dripping concentrated ammonia slowly, and stirring to react for 10-30 minutes;
after reaction ends, standing reaction liquid at 0-10° C. for 4-6 hours;
washing by using methanol, adding a methanol solution of oleic acid, and stirring for 2-5 hours, to obtain the magnetic particles; and
re-washing by using methanol, and dispersing the obtained magnetic particles into hexane.

20. The preparing method according to claim 14, wherein the step of forming the magnetic particles comprises:
mixing ferric trichloride, ferrous chloride, hydrochloric acid and deionized water together, and stirring uniformly, to obtain an iron-ion mixture liquid;
introducing a protective gas, dripping the iron-ion mixture liquid into a sodium-hydroxide solution at 80° C.±5° C. under continuous stirring, and after the iron-ion mixture liquid is dripped completely, reacting for 20-40 minutes; and
after reaction ends, cooling to room temperature, washing by using anhydrous ethanol and deionized water, and drying to obtain the magnetic particles.
